(19) United States
(12) Patent Application Publication
(10) Pub. No.: US 2026/0294529 A1
(43) Pub. Date: Oct. 1, 2026

(54) IMPEDANCE BASED METRIC OF TISSUE COUPLING

(71) Applicant: Boston Scientific Scimed, Inc., Maple Grove, MN (US)

(72) Inventors: Sarah Renee Gutbrod, Medford, MA (US); Petar Yosifov Dimitrov, Arlington, MA (US); Jaume Coll-Font, Cambridge, MA (US); Jason Michael Clevenger, Needham, MA (US); Matthew D. Ferrante, Cambridge, MA (US)

(21) Appl. No.: 19/631,132

(22) Filed: Mar. 27, 2026

Related U.S. Application Data
(60) Provisional application No. 63/793,483, filed on Apr. 23, 2025.
(60) Provisional application No. 63/780,020, filed on Mar. 28, 2025.

(51) Int. Cl. A61B 18/14; A61B 34/10; A61B 17/00; A61B 18/00

(52) U.S. Cl. CPC A61B 18/1492; A61B 34/10; A61B 2017/00199; A61B 2018/00357; A61B 2018/00577; A61B 2018/00613; A61B 2034/104; A61B 2034/105

(57) ABSTRACT

A system for irreversible electroporation of cardiac tissue comprises a catheter having electrode assembly including an ablation electrode and mapping electrodes. One or more controllers are configured to generate and display, on a graphical display, an anatomical model of a cardiac chamber and a graphical representation of the electrode assembly, generate a model of an electric field generated in response to delivery of a pulse waveform to the ablation electrode, generate and display, on the graphical display, a graphical representation of a predicted ablation zone on the anatomical model of the cardiac chamber based on the model of the electric field and the determined proximity of the electrode assembly to the cardiac tissue surface, and generate and display, on the graphical display, one or more visual indicators of tissue coupling responsive to the determined proximity of the electrode assembly to the cardiac tissue satisfying a pre-determined criteria.

### CROSS REFERENCE TO RELATED APPLICATIONS

This application claims priority to U.S. Provisional Patent Application No. 63/780,020 entitled “IMPEDANCE BASED METRIC OF TISSUE COUPLING,” filed Mar. 28, 2025, and claims priority to U.S. Provisional Patent Application No. 63/793,483 entitled “IMPEDANCE BASED METRIC OF TISSUE COUPLING,” filed Apr. 23, 2025, which are hereby incorporated by reference in their entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a system for ablating cardiac tissue by irreversible electroporation, the system comprising a catheter, a graphical display, and one or more controllers. The catheter has a tubular outer shaft having a proximal end and an opposite distal end; and an electrode assembly extending distally from the distal end of the outer shaft, the electrode assembly comprising a plurality of splines forming a basket shape when in an expanded configuration, an ablation electrode disposed on the splines, and a set of mapping electrodes disposed along each spline of the plurality of splines. The one or more controllers are configured to generate and display, on the graphical display, an anatomical model of a cardiac chamber and a graphical representation of the electrode assembly, determine a proximity of the electrode assembly to the cardiac tissue surface, generate a model of an electric field generated in response to delivery of a pulse waveform to the ablation electrode, generate and display, on the graphical display, a graphical representation of a predicted ablation zone on the anatomical model of the cardiac chamber based on one or both of the model of the electric field and the determined proximity of the electrode assembly to the cardiac tissue surface, and generate and display, on the graphical display, one or more visual indicators of tissue coupling responsive to the determined proximity of the electrode assembly to the cardiac tissue satisfying a pre-determined criteria.

In Example 2, the system of Example 1, wherein the predicted ablation zone is based on an intersection of the modeled electric field with the cardiac tissue surface on the anatomical model of the cardiac chamber.

In Example 3, the system of either of Examples 1 or 2, wherein the one or more visual indicators provides a visual indicator of a degree of contact between at least a portion of the electrode assembly and the cardiac tissue surface.

In Example 4, the system of any of Examples 1-3, wherein the pre-determined criteria is based on a pre-determined number of the mapping electrodes being determined to be in contact with the cardiac tissue surface.

In Example 5, the system of any of Examples 1-4, wherein the one or more visual indicators comprises a visual indicator disposed on at least a portion of the graphical representation of the electrode assembly.

In Example 6, the system of any of Examples 1-4, wherein the one or more visual indicators comprises an altered visual of at least a portion of the graphical representation of the electrode assembly based on the determined proximity of the electrode assembly to the cardiac tissue surface.

In Example 7, the system of any of Examples 1-6, wherein the one or more visual indicators comprises a border circumscribing the graphical representation of the predicted ablation zone on the anatomical model of the cardiac chamber.

In Example 8, the system of any of Examples 1-7, wherein the one or more controllers are further configured to automatically annotate the anatomical model with an ablation marker corresponding to the predicted ablation zone upon completion of delivery of the pulse waveform to the ablation electrode.

In Example 9, an apparatus for use in ablation of cardiac tissue by irreversible electroporation, the apparatus comprising a graphical display, one or more controllers configured to generate and display, on the graphical display, an anatomical model of a cardiac chamber and a graphical representation of an electrode assembly of an ablation catheter, determine a proximity of the electrode assembly to the cardiac tissue surface, generate a model of an electric field generated in response to delivery of a pulse waveform to the electrode assembly, generate and display, on the graphical display, a graphical representation of a predicted ablation zone on the anatomical model of the cardiac chamber based on one or both of the model of the electric field and the determined proximity of the electrode assembly to the cardiac tissue surface, and generate and display, on the graphical display, one or more visual indicators of tissue coupling responsive to the determined proximity of the electrode assembly to the cardiac tissue satisfying a pre-determined criteria.

In Example 10, the apparatus of Example 9, wherein the predicted ablation zone is based on an intersection of the modeled electric field with the cardiac tissue surface on the anatomical model of the cardiac chamber.

In Example 11, the apparatus of either of Examples 9 or 10, wherein the electrode assembly includes a plurality of mapping electrodes, and the pre-determined criteria is based on a pre-determined number of the mapping electrodes being determined to be in contact with the cardiac tissue surface.

In Example 12, the apparatus of any of Examples 9-11, wherein the one or more visual indicators comprise a visual indicator disposed on at least a portion of the graphical representation of the electrode assembly.

In Example 13, the apparatus of any of Examples 9-11, wherein the one or more visual indicators comprise an altered graphical representation of at least a portion of the graphical representation based on the determined proximity of the electrode assembly to the cardiac tissue surface.

In Example 14, the apparatus of any of Examples 9-13, wherein the one or more visual indicators comprise a border circumscribing the graphical representation of the predicted ablation zone on the anatomical model of the cardiac chamber.

In Example 15, the apparatus of any of Examples 9-14, wherein the one or more controllers are further configured to automatically annotate the anatomical model with an ablation marker corresponding to the predicted ablation zone upon completion of delivery of the pulse waveform to the ablation electrode.

In Example 16, a system for ablating cardiac tissue by irreversible electroporation, the system comprising a catheter, a graphical display, and one or more controllers. The catheter has a tubular outer shaft having a proximal end and an opposite distal end, and an electrode assembly extending distally from the distal end of the outer shaft, the electrode assembly comprising one or more electrodes. The one or more controllers are configured to generate and display, on the graphical display, an anatomical model of a cardiac chamber and a graphical representation of the electrode assembly, determine a proximity of the electrode assembly to the cardiac tissue surface, generate a model of an electric field generated in response to delivery of a pulse waveform to the one or more electrodes, generate and display, on the graphical display, a graphical representation of a predicted ablation zone on the anatomical model of the cardiac chamber based on one or both of the model of the electric field and the determined proximity of the electrode assembly to the cardiac tissue surface, and generate and display, on the graphical display, one or more visual indicators of tissue coupling responsive to the determined proximity of the electrode assembly to the cardiac tissue satisfying a pre-determined criteria.

In Example 17, the system of Example 16, wherein the predicted ablation zone is based on an intersection of the modeled electric field with the cardiac tissue surface on the anatomical model of the cardiac chamber.

In Example 18, the system of Example 16, wherein the one or more visual indicators comprise a visual indicator of a degree of contact between at least a portion of the electrode assembly and the cardiac tissue surface.

In Example 19, the system of Example 18, wherein the pre-determined criteria is based on a pre-determined number of the one or more electrodes being determined to be in contact with the cardiac tissue surface.

In Example 20, the system of Example 16, wherein the one or more visual indicators comprise a visual indicator disposed on at least a portion of the graphical representation of the electrode assembly.

In Example 21, the system of Example 16, wherein the one or more visual indicators comprise an altered graphical representation of at least a portion of the electrode assembly based on the determined proximity of the electrode assembly to the cardiac tissue surface.

In Example 22, the system of Example 16, wherein the one or more visual indicators comprise a border circumscribing the graphical representation of the predicted ablation zone on the anatomical model of the cardiac chamber.

In Example 23, the system of Example 22, wherein the one or more controllers are further configured to automatically annotate the anatomical model with an ablation marker corresponding to the predicted ablation zone upon completion of delivery of the pulse waveform to the ablation electrode.

In Example 24, an apparatus for use in ablation of cardiac tissue by irreversible electroporation, the apparatus comprising a graphical display, and one or more controllers configured to generate and display, on the graphical display, an anatomical model of a cardiac chamber and a graphical representation of an electrode assembly of an ablation catheter, the electrode assembly including one or more electrodes, determine a proximity of the electrode assembly to the cardiac tissue surface, generate a model of an electric field generated in response to delivery of a pulse waveform to the one or more electrodes, generate and display, on the graphical display, a graphical representation of a predicted ablation zone on the anatomical model of the cardiac chamber based on one or both of the model of the electric field and the determined proximity of the electrode assembly to the cardiac tissue surface, and generate and display, on the graphical display, one or more visual indicators of tissue coupling responsive to the determined proximity of the electrode assembly to the cardiac tissue satisfying a pre-determined criteria.

In Example 25, the apparatus of Example 24, wherein the predicted ablation zone is based on an intersection of the modeled electric field with the cardiac tissue surface on the anatomical model of the cardiac chamber.

In Example 26, the apparatus of Example 25, wherein the pre-determined criteria is based on a pre-determined number of the one or more electrodes being determined to be in contact with the cardiac tissue surface.

In Example 27, the apparatus of Example 26, wherein the one or more visual indicators comprise a visual indicator disposed on at least a portion of the graphical representation of the electrode assembly.

In Example 28, the apparatus of Example 26, wherein the one or more visual indicators comprise an altered graphical representation of at least a portion of the electrode assembly based on the determined proximity of the electrode assembly to the cardiac tissue surface.

In Example 29, the apparatus of Example 26, wherein the one or more visual indicators comprise a border circumscribing the graphical representation of the predicted ablation zone on the anatomical model of the cardiac chamber.

In Example 30, the apparatus of Example 26, wherein the one or more controllers are further configured to automatically annotate the anatomical model with an ablation marker corresponding to the predicted ablation zone upon completion of delivery of the pulse waveform to the ablation electrode.

In example 31, a method of graphically representing an ablation procedure on a graphical display, the method comprising generating and displaying, on the graphical display, an anatomical model of a cardiac chamber and a graphical representation of an electrode assembly of an ablation catheter, the electrode assembly including one or more electrodes, determining a proximity of the electrode assembly to the cardiac tissue surface, generating a model of an electric field generated in response to delivery of a pulse waveform to the one or more electrodes, generating and displaying, on the graphical display, a graphical representation of a predicted ablation zone on the anatomical model of the cardiac chamber based on one or both of the model of the electric field and the determined proximity of the electrode assembly to the cardiac tissue surface, and generating and displaying, on the graphical display, one or more visual indicators of tissue coupling responsive to the determined proximity of the electrode assembly to the cardiac tissue satisfying a pre-determined criteria.

In Example 32, the method of Example 31, wherein the pre-determined criteria is based on a pre-determined number of the one or more electrodes being determined to be in contact with the cardiac tissue surface.

In Example 33, the method of Example 32, wherein the one or more visual indicators comprise a visual indicator disposed on at least a portion of the graphical representation of the electrode assembly.

In Example 34, the method of Example 32, wherein the one or more visual indicators comprise an altered graphical representation of at least a portion of the electrode assembly based on the determined proximity of the electrode assembly to the cardiac tissue surface.

In Example 35, the method of Example 32, wherein the one or more visual indicators comprise a border circumscribing the graphical representation of the predicted ablation zone on the anatomical model of the cardiac chamber.While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is a perspective illustration of a distal portion of a splined catheter for use in the electrophysiology system of FIG. 1, in accordance with embodiments of the subject matter of the disclosure.

FIGS. 3A-3C illustrate an exemplary display window of a graphical display of the electrophysiology system of FIG. 1 depicting a graphical representation of an electrode assembly of an ablation catheter and an anatomical model of a cardiac chamber, in accordance with embodiments of the subject matter of the disclosure.

FIGS. 4A-4B illustrate an exemplary display window of a graphical display of the electrophysiology system of FIG. 1, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

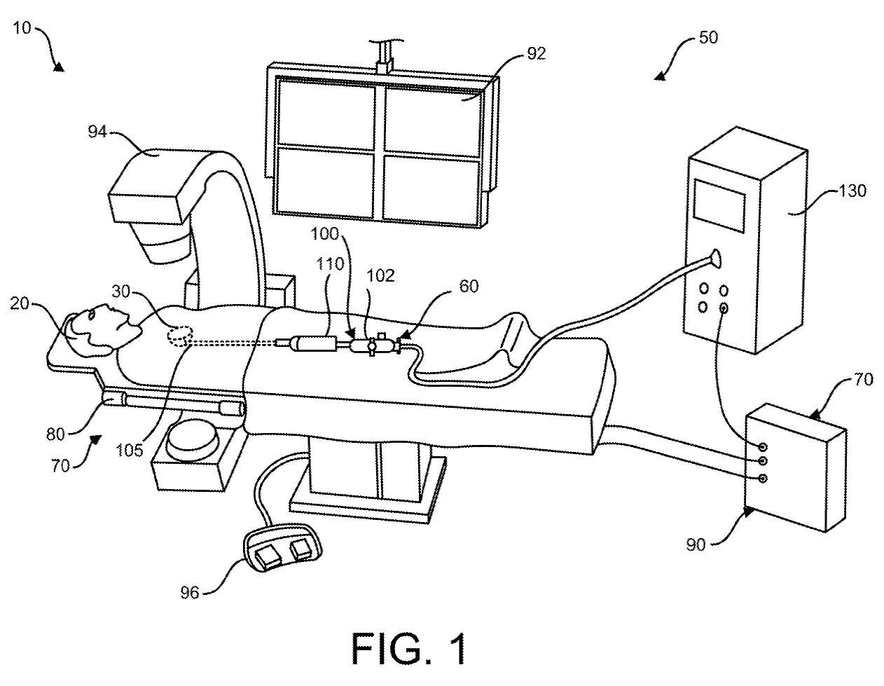

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an ablation catheter system 60 and an electro-anatomical mapping (EAM) system 70, which includes a localization field generator 80, a mapping and navigation controller 90, and a graphical display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The ablation catheter system 60 includes an ablation catheter 100 having a proximal portion including a handle 102 and a distal portion 105, an introducer sheath 110, and an electroporation console 130. Additionally, the ablation catheter system 60 includes various connecting elements, e.g., cables, umbilicals, and the like, that operate to functionally connect the components of the ablation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the ablation catheter 100, in particular all or part of the distal portion 105 thereof, can be deployed to the specific target sites within the patient’s heart 30.

In embodiments, the ablation catheter system 60 is configured to deliver electric field energy to targeted tissue in the patient’s heart 30 to create tissue apoptosis, rendering the tissue incapable of conducting electrical signals.

The electroporation console 130 is configured to control functional aspects of the ablation catheter system 60. In embodiments, the electroporation console 130 includes one or more controllers, microprocessors, and/or computers that execute code out of memory to control and/or perform the functional aspects of the ablation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the ablation catheter 100 to generate electric fields sufficient to achieve the desired clinical effect, in particular ablation of target tissue through irreversible electroporation. In embodiments, the electroporation console 130 can deliver the pulsed waveforms to the ablation catheter 100 in a monopolar or bipolar mode of operation, as will be described in further detail herein.

The EAM system 70 is operable to track the location of the various functional components of the ablation catheter system 60, and to generate high-fidelity three-dimensional anatomical and electro-anatomical maps of the cardiac chambers of interest. In embodiments, the EAM system 70 can be the OPAL HDx™ mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the ablation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the ablation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

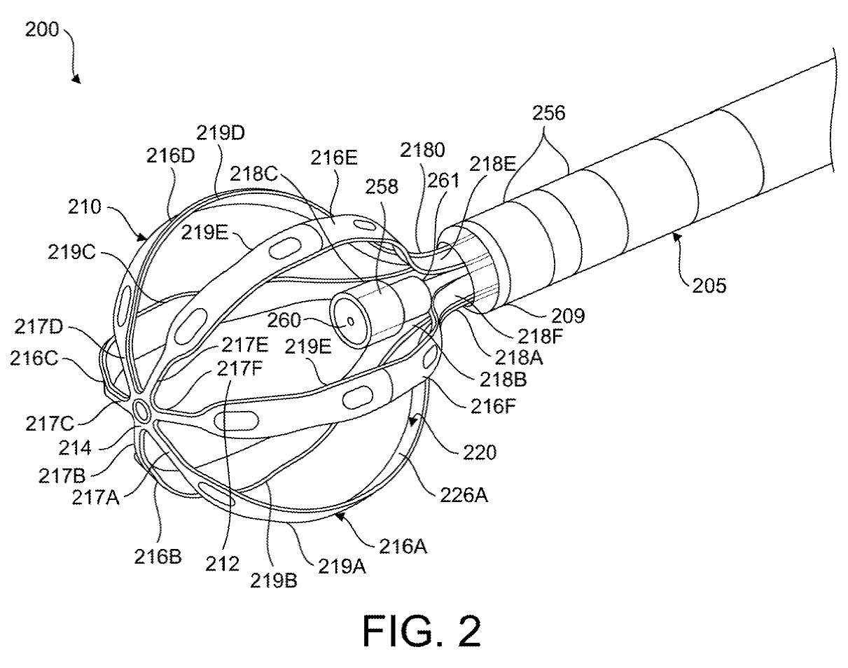

FIG. 2 is a partial perspective view illustration of an ablation catheter 200 having a catheter distal portion 205 according to an embodiment of the present disclosure. The ablation catheter 200 corresponds to the ablation catheter 100 described with respect to FIG. 1. The ablation catheter 200 has a tubular outer shaft 202 having a shaft distal end 209, and an electrode assembly 210 extending distally from the distal end 209 of the outer shaft 202. In embodiments, the electrode assembly 210 is configured to self-expand from a collapsed configuration when constrained within a delivery sheath to a pre-defined expanded configuration as depicted in FIG. 2. As will be explained in greater detail herein, the electrode assembly 210 comprises an ablation electrode configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, are designed for the creation of relatively localized ablation lesions (i.e., focal lesions), as well as relatively large diameter circumferential lesions created in pulmonary vein isolation procedures. The designs of the various electrode assembly embodiments described herein can provide the clinician with a wide range of capabilities for monopolar and bipolar pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

In various embodiments, the ablation catheter 200 may correspond to any of the embodiments of ablation catheters illustrated and described in commonly-assigned U.S. Patent Application No. 18/634,785, the entire disclosure of which is incorporated herein by reference. In other embodiments, the ablation catheter 200 may correspond to any of the endocardial pulsed field ablation catheters disclosed in commonly-assigned U.S. Patent Nos. 10,172,673 and 10,617,467 and 10,020,179, the entire disclosures of which are incorporated herein by reference. Additionally, the present disclosure is fully applicable to the commercially-available FARAWAVE™ pulsed field ablation catheter marketed by Boston Scientific Corporation.

As shown in FIG. 2, in the illustrated embodiment, the electrode assembly 210 has a plurality of splines 216A-216F extending proximally from a central hub portion 214. In embodiments, the particular geometry of the splines 216A-216F and the related components, e.g., ablation and mapping electrodes, is optimized to provide desired mechanical and therapeutic/diagnostic capabilities. In the illustrated embodiment, the electrode assembly 210 includes an ablation electrode 238 and a plurality of mapping electrodes 250 disposed on the splines 216A-216F (for ease of illustration purposes, only a single mapping electrode 250 is shown in FIG. 2). Additionally, the illustrated embodiment includes a central post 258 which may, when present, provide additional functionality, including for example, housing or supporting a unipolar reference electrode and/or a magnetic position sensor.

It will be appreciated by the skilled artisan that while the embodiment illustrated in FIG. 2 includes separate ablation and mapping electrodes 238, 250, respectively, in other embodiments, one or more electrodes may be configured to be suitable for both ablation and mapping functionality.

In use, the effectiveness of the electrophysiology system 50 in ablating target tissue can be significantly affected by the proximity of the ablation electrode 238 to the tissue, e.g., the endocardial surface of the heart. This is due to the fact that electric field strength decreases with distance away from the source of the electric field (i.e., the ablation electrode 238). While actual physical contact between the ablation electrode 238 and the cardiac tissue is not strictly necessary to create effective ablation lesions, when the ablation electrode 238 is in intimate contact and thereby coupled to the target tissue, the tissue will be exposed to the maximum electric field strength for a given set of pulse waveform parameters, e.g., voltage amplitude. Additionally, the lesion depth into the tissue will be maximized when the ablation electrode 238 is in contact with the tissue.

Accordingly, the electrophysiology system 50 is configured to include a tissue coupling capability so as to determine if the ablation electrode 238 is (or is likely to be) in contact with the cardiac tissue. This capability is enabled, in part, by the EAM system 70, which can track the position of the electrode assembly 210 via, for example, a magnetic sensor (not shown) located within the central post 258, by impedance tracking of the ablation electrode 238 and/or the mapping electrodes 250, or combinations thereof.

Additionally, a controller of the electroporation console 130 is configured to determine the proximity of the electrode assembly 210, and thus the ablation electrode 238, to the target tissue, including whether or not the ablation electrode 238 is, or is likely to be, in sufficient contact with the target tissue to maximize the effectiveness of the delivery of electroporation energy. In embodiments, the controller utilizes local impedance techniques whereby relatively low-amplitude electric signals are driven between certain electrodes (e.g., selected mapping electrodes 250) on the ablation catheter 200, and a response voltage is measured at the same or different electrodes. Such techniques are described, for example, in commonly-assigned U.S. Patent Application No. 18/991,089, the disclosure of which is incorporated herein by reference. In such embodiments, coupling between the ablation electrode 238 and the target tissue can be assessed by the controller based on the number of the mapping electrodes 250 that re determined to be in contact with the tissue. However, it is emphasized that the particular technique or methodology used for electrode-tissue coupling assessment is not critical to the present disclosure, and thus other such techniques may be employed by the electrophysiology system 50.

In the various embodiments, the electrophysiology system 50 is further configured to provide, via the graphical display 92 (see FIG. 1), visual information regarding the assessed proximity of the ablation electrode 238 and the target tissue. In addition, the controller of the electroporation console 130 is configured to model the electric fields generated upon delivery of the pulse waveform to the ablation electrode 238, and to annotate the anatomical model of the target cardiac region upon completion of delivery of the ablative energy, such as is described, for example, in commonly-assigned U.S. Patent Application No. 18/510,087, the entire disclosure of which is incorporated herein by reference. Accordingly, the electrophysiology system 50 is configured to provide the clinician with multiple levels of information to aid in ablation procedure planning and execution, and for assessment of the effectiveness of the ablation procedure.

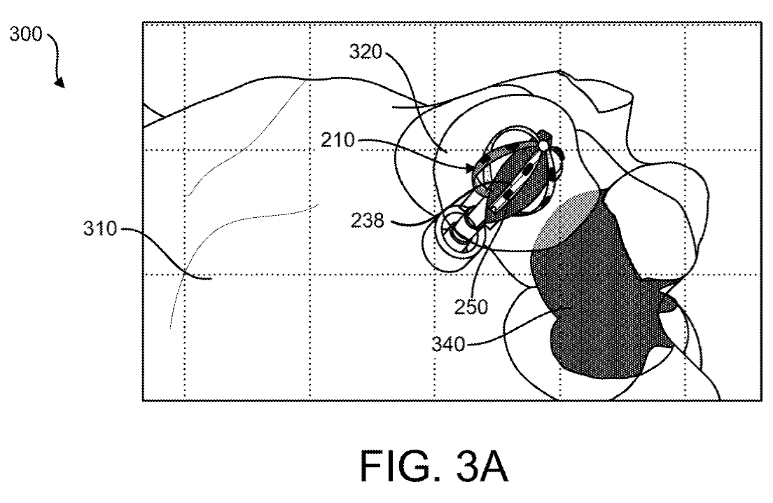

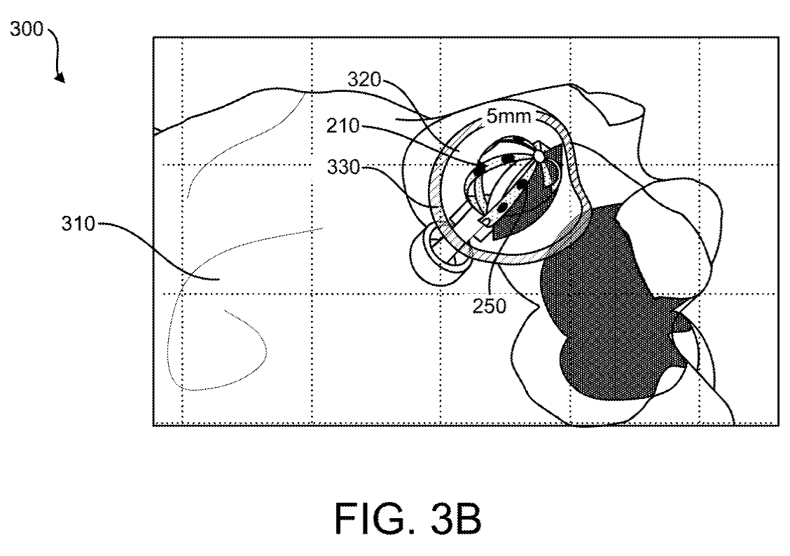

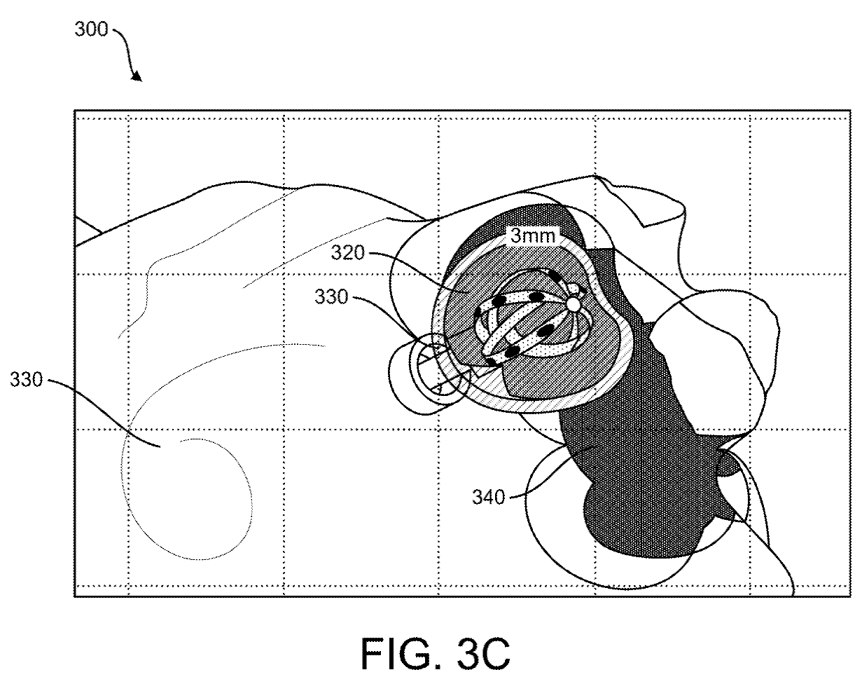

FIGS. 3A-3C illustrate an exemplary display window 300 of the graphical display 92 (FIG. 1) depicting a graphical representation of the electrode assembly 210 and an anatomical model 310 of a cardiac chamber, e.g., the left atrium, with the electrode assembly 210 at various exemplary positions within the cardiac chamber and at various stages of a pulsed field ablation procedure. As described herein above, the controller of the electroporation console 130 is configured to generate a model of the electric field generated in response to delivery of a pulse waveform to the ablation electrode 238. Additionally, the electrophysiology system 50 (FIG. 1) (i.e., via one or more controllers thereof) is configured determine the proximity of the electrode assembly 210, and consequently, the ablation electrode 238, to the cardiac tissue surface represented by the anatomical model 310. Accordingly, as shown in FIGS. 3A-3C, the one or more controllers are configured to generate and display, on the display window 300, a graphical representation of a predicted ablation zone 320 based on one or both of the model of the electric field and the determined proximity of the electrode assembly to the cardiac tissue surface represented by the anatomical model 310. In some embodiments, the predicted ablation zone 320 is determined based on an intersection of the modeled electric field with the cardiac tissue surface, as described, for example, in the above-mentioned U.S. Patent Application No. 18/510,087, which is incorporated herein by reference in its entirety.

In the various embodiments, the one or more controllers of electrophysiology system 50 are further configured to generate and display, on the display window 300, some visual indicator(s) of tissue coupling responsive to the determined proximity of the electrode assembly 210, and consequently, the ablation electrode 238, to the cardiac tissue represented by the anatomical model 310. In embodiments, the visual indicator(s) provide an indicator of a degree of contact between at least a portion of the electrode assembly 210 and the cardiac tissue surface. In some embodiments, the visual indicator(s) are displayed upon the determined proximity of the electrode assembly 210 to the cardiac tissue satisfying a pre-determined criteria. In embodiments, the pre-determined criteria may be based on a pre-determined number or distribution of the mapping electrodes 250 being determined by the one or more controllers to be in contact with the cardiac tissue surface.

In some embodiments, the visual indicator(s) of tissue coupling may take the form of a visual indicator on the graphical representation of the electrode assembly 210. For example, in embodiments, the electrode assembly 210 (or portions thereof determined to be in contact with the cardiac tissue) may be highlighted or otherwise depicted in a different manner when determined to be coupled to tissue than when no tissue coupling is determined. In embodiments, at least a portion of the graphical representation of the electrode assembly 210 is altered based on the portion(s) determined to be in contact with the cardiac tissue surface. For example, in some embodiments, the mapping electrodes 250 that are determined to be in contact with the cardiac tissue surface are depicted differently (e.g., via coloration, shading, and the like) than those that are not determined to be tissue-contacting.

In embodiments, the visual indicator(s) of tissue coupling comprise a border 330 (FIG. 3B) circumscribing the graphical representation of the predicted ablation zone 320.

Additionally, in the various embodiments, the one or more controllers are configured to automatically annotate the anatomical model 310 with an ablation marker (e.g., the ablation makers 340) upon completion of the application of ablation energy at a particular location relative to the cardiac tissue. As can be seen, for example, in FIG. 3A, the ablation markers 340 have a different visual appearance (e.g., coloration) than the predicted ablation zone 320, to aid the clinician in positioning the electrode assembly 210 for the current application of energy, as described, for example, in the above-referenced U.S. Patent Application No. 18/510,087.

Referring again to FIGS. 3A-3C, FIG. 3A depicts a condition in which the electrode assembly 210 is determined not to be sufficiently coupled to the cardiac tissue surface so as to trigger the inclusion of an indicator of tissue coupling within the display window 300. In contrast, in the state depicted in FIG. 3B, the electrode assembly 210 has been moved to a position such that it is determined to be sufficiently coupled to the tissue based on the pre-determined criteria described above. As such, the border 330 has been applied circumscribing the predicted ablation zone 320. Additionally, the electrophysiology system 50 has further altered the visual appearance of the ablation assembly relative to its depiction in FIG. 3A. One or both of these visual indicia can be used by the clinician to aid in procedure planning and assessment. As can further be seen with particular reference to FIG. 3C, the system 50 enables the clinician to visualize the degree of overlap of the predicted ablation zone 320 with an existing ablation marker 340. The system 50 can optionally provide an indication (e.g., a numerical value) between approximate centers of the predicted ablation zone 320 and a nearby ablation marker 340. And as previously discussed, the indicator of tissue coupling (e.g., the border 330 circumscribing the predicted ablation zone 320) provides a visual indication that lesion depth will be maximized upon the application of ablative energy. The system 50 thus provides multiple levels of visual confirmation of desired positioning of the electrode assembly for the application of the ablative pulse waveform.

Additionally, in the various embodiments, the one or more controllers are configured to automatically annotate the anatomical model 310 with an ablation marker (e.g., the ablation makers 340) upon completion of the application of ablation energy at a particular location relative to the cardiac tissue. As can be seen, for example, in FIG. 3A, the ablation markers 340 have a different visual appearance (e.g., coloration) than the predicted ablation zone 320, to aid the clinician in positioning the electrode assembly 210 for the current application of energy, as described, for example, in the above-referenced U.S. Patent Application No. 18/510,087.

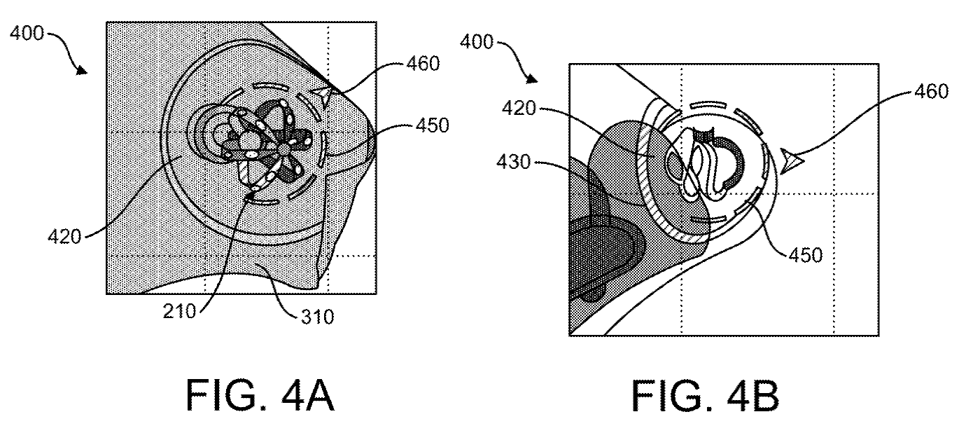

FIGS. 4A-4B illustrate an exemplary display window 400 of the graphical display 92 (FIG. 1) depicting a graphical representation of the electrode assembly 210 and an anatomical model 410 of a cardiac chamber, e.g., the left atrium, with the electrode assembly 210 at various exemplary positions within the cardiac chamber and at various stages of a pulsed field ablation procedure. As with the embodiments of FIGS. 3A-3C, the controller of the electroporation console 130 is configured to generate a model of the electric field generated in response to delivery of a pulse waveform to the electrode assembly 210. Additionally, the electrophysiology system 50 (FIG. 1) (i.e., via one or more controllers thereof) is configured determine the proximity of the electrode assembly 210, and consequently, the ablation electrode(s) thereon, to the cardiac tissue surface represented by the anatomical model 410. Accordingly, as shown in FIGS. 4A-4B, the one or more controllers are configured to generate and display, on the display window 400, a graphical representation of a predicted ablation zone 420 based on one or both of the model of the electric field and the determined proximity of the electrode assembly to the cardiac tissue surface represented by the anatomical model 410. In some embodiments, the predicted ablation zone 420 is determined based on an intersection of the modeled electric field with the cardiac tissue surface, as described, for example, in the above-mentioned U.S. Patent Application No. 18/510,087, which is incorporated herein by reference in its entirety.

In the various embodiments, the one or more controllers of electrophysiology system 50 are further configured to generate and display, on the display window 400, some visual indicator(s) of tissue coupling responsive to the determined proximity of the electrode assembly 210 to the cardiac tissue represented by the anatomical model 310. In embodiments, the visual indicator(s) provide an indicator of a degree of contact between at least a portion of the electrode assembly 210 and the cardiac tissue surface. In some embodiments, the visual indicator(s) are displayed upon the determined proximity of the electrode assembly 210 to the cardiac tissue satisfying a pre-determined criteria. For example, in some embodiments, the portions of the electrode assembly 210 determined to be in contact with the cardiac tissue surface are depicted differently (e.g., via coloration, shading, and the like) than those that are not determined to be tissue-contacting. Additionally, or alternatively, in embodiments, the visual indicator(s) of tissue coupling comprise a border 430 (FIG. 4B) circumscribing the graphical representation of the predicted ablation zone 420.

As further shown in FIGS. 4A-4B, an additional indicator 450 is provided in proximity to the region(s) of the electrode assembly 210 determined to be in contact with tissue. In the illustrated embodiment, the additional indicator 450 is in the form of a semi-circular dashed arc. In embodiments, the length of the arc can provide a visual indication of the magnitude or extent of contact between the electrode assembly 210 and the tissue. As further shown in FIGS. 4A-4B, the controller(s) of the electrophysiology system 50 are configured to provide a directional proximity indicator 460 on the display window 400, which provides a visual indicator to the user of the direction of contact or closest proximity between the electrode assembly 210 and the target tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for ablating cardiac tissue by irreversible electroporation, the system comprising:
a catheter having a tubular outer shaft having a proximal end and an opposite distal end, and an electrode assembly extending distally from the distal end of the outer shaft, the electrode assembly comprising one or more electrodes;
a graphical display; and
one or more controllers configured to:
generate and display, on the graphical display, an anatomical model of a cardiac chamber and a graphical representation of the electrode assembly;
determine a proximity of the electrode assembly to the cardiac tissue surface;
generate a model of an electric field generated in response to delivery of a pulse waveform to the one or more electrodes;
generate and display, on the graphical display, a graphical representation of a predicted ablation zone on the anatomical model of the cardiac chamber based on one or both of the model of the electric field and the determined proximity of the electrode assembly to the cardiac tissue surface; and
generate and display, on the graphical display, one or more visual indicators of tissue coupling responsive to the determined proximity of the electrode assembly to the cardiac tissue satisfying a pre-determined criteria.

2. The system of claim 1, wherein the predicted ablation zone is based on an intersection of the modeled electric field with the cardiac tissue surface on the anatomical model of the cardiac chamber.

3. The system of claim 1, wherein the one or more visual indicators comprise a visual indicator of a degree of contact between at least a portion of the electrode assembly and the cardiac tissue surface.

4. The system of claim 3, wherein the pre-determined criteria is based on a pre-determined number of one or more electrodes being determined to be in contact with the cardiac tissue surface.

5. The system of claim 1, wherein the one or more visual indicators comprise a visual indicator disposed on at least a portion of the graphical representation of the electrode assembly.

6. The system of claim 1, wherein the one or more visual indicators comprise an altered graphical representation of at least a portion of the electrode assembly based on the determined proximity of the electrode assembly to the cardiac tissue surface.

7. The system of claim 1, wherein the one or more visual indicators comprise a border circumscribing the graphical representation of the predicted ablation zone on the anatomical model of the cardiac chamber.

8. The system of claim 7, wherein the one or more controllers are further configured to automatically annotate the anatomical model with an ablation marker corresponding to the predicted ablation zone upon completion of delivery of the pulse waveform to the ablation electrode.

9. An apparatus for use in ablation of cardiac tissue by irreversible electroporation, the apparatus comprising:
a graphical display; and
one or more controllers configured to:
generate and display, on the graphical display, an anatomical model of a cardiac chamber and a graphical representation of an electrode assembly of an ablation catheter, the electrode assembly including one or more electrodes;
determine a proximity of the electrode assembly to the cardiac tissue surface;
generate a model of an electric field generated in response to delivery of a pulse waveform to the one or more electrodes;
generate and display, on the graphical display, a graphical representation of a predicted ablation zone on the anatomical model of the cardiac chamber based on one or both of the model of the electric field and the determined proximity of the electrode assembly to the cardiac tissue surface; and
generate and display, on the graphical display, one or more visual indicators of tissue coupling responsive to the determined proximity of the electrode assembly to the cardiac tissue satisfying a pre-determined criteria.

10. The apparatus of claim 9, wherein the predicted ablation zone is based on an intersection of the modeled electric field with the cardiac tissue surface on the anatomical model of the cardiac chamber.

11. The apparatus of claim 10, wherein the pre-determined criteria is based on a pre-determined number of one or more electrodes being determined to be in contact with the cardiac tissue surface.

12. The apparatus of claim 11, wherein the one or more visual indicators comprise a visual indicator disposed on at least a portion of the graphical representation of the electrode assembly.

13. The apparatus of claim 11, wherein the one or more visual indicators comprise an altered graphical representation of at least a portion of the electrode assembly based on the determined proximity of the electrode assembly to the cardiac tissue surface.

14. The apparatus of claim 11, wherein the one or more visual indicators comprise a border circumscribing the graphical representation of the predicted ablation zone on the anatomical model of the cardiac chamber.

15. The apparatus of claim 11, wherein the one or more controllers are further configured to automatically annotate the anatomical model with an ablation marker corresponding to the predicted ablation zone upon completion of delivery of the pulse waveform to the ablation electrode.

16. A method of graphically representing an ablation procedure on a graphical display, the method comprising:
generating and displaying, on the graphical display, an anatomical model of a cardiac chamber and a graphical representation of an electrode assembly of an ablation catheter, the electrode assembly including one or more electrodes;
determining a proximity of the electrode assembly to the cardiac tissue surface;
generating a model of an electric field generated in response to delivery of a pulse waveform to the one or more electrodes;
generating and displaying, on the graphical display, a graphical representation of a predicted ablation zone on the anatomical model of the cardiac chamber based on one or both of the model of the electric field and the determined proximity of the electrode assembly to the cardiac tissue surface; and
generating and displaying, on the graphical display, one or more visual indicators of tissue coupling responsive to the determined proximity of the electrode assembly to the cardiac tissue satisfying a pre-determined criteria.

17. The method of claim 16, wherein the pre-determined criteria is based on a pre-determined number of one or more electrodes being determined to be in contact with the cardiac tissue surface.

18. The method of claim 17, wherein the one or more visual indicators comprise a visual indicator disposed on at least a portion of the graphical representation of the electrode assembly.

19. The method of claim 17, wherein the one or more visual indicators comprise an altered graphical representation of at least a portion of the electrode assembly based on the determined proximity of the electrode assembly to the cardiac tissue surface.

20. The method of claim 17, wherein the one or more visual indicators comprise a border circumscribing the graphical representation of the predicted ablation zone on the anatomical model of the cardiac chamber.
